(19) United States
(12) Patent Application Publication
(10) Pub. No.: US 2024/0016834 A1
(43) Pub. Date: Jan. 18, 2024

(54) ANTIMICROBIAL AGENT COMPOSITION AND MANUFACTURING METHOD AND PRODUCT THEREOF

(71) Applicants: Woo Jun KIM, Icheon-si (KR); Dong Chul IHN, Yongin-si (KR)

(72) Inventor: Dong Chul IHN, Yongin-si (KR)

(21) Appl. No.: 18/258,456

(22) PCT Filed: Oct. 7, 2021
(86) PCT No.: PCT/KR2021/013739

§ 371 Date: Jun. 20, 2023

(30) Foreign Application Priority Data
Dec. 24, 2020 (KR) 10-2020-0183573

(51) Int. Cl. A61K 33/14; A61K 33/06; A61K 36/488; A61K 36/34; A61K 33/20; A61K 31/191; A61P 31/04

(52) U.S. Cl. CPC A61K 33/14; A61K 33/06; A61K 36/488; A61K 36/34; A61K 33/20; A61K 31/191; A61P 31/04

(57) ABSTRACT

The present invention relates to an antimicrobial agent composition comprising an undiluted mixture solution formed by mixing: 1 to 10 wt % of a mixture comprising 50 to 60 wt % of purified water and chloride in which sodium chloride and calcium chloride which are at a 1:1 ratio are contained; 1 to 2 wt % of a mixture comprising Pueraria lobata root extract and bellflower root extract which are selected from among natural extracts and are at a 1:1 ratio; 5 to 10 wt % of a mixture comprising carbonate based material in which sodium carbonate and sodium bicarbonate which are at a 1:1 ratio are contained; 10 to 15 wt % of a mixture comprising lactic acid and acetic acid among organic acids and their related salts, which are at a 1:1 ratio; and 5 to 10 wt % of chloric acid based compound of inorganic acid.

### FIELD OF THE INVENTION

The present invention relates to an antimicrobial agent composition, and a manufacturing method and a product thereof, and more particularly, to an antimicrobial agent composition, and a manufacturing method and a product thereof which are formulated with natural extract, organic acid with antibacterial or antiviral activity, carbonate compound, and chlorine based compound.

In addition, an object of the composition of the present invention are to achieve an antibacterial activity effect against major bacteria which generally cause food poisoning, such as colon Bacillus and Salmonella, and Staphylococcus aureus, which causes skin suppuration, and to achieve an antiviral effect against envelop viruses such as herpesvirus, coronavirus, and influenza.

### BACKGROUND OF THE INVENTION

The rapid increase in antibiotic-resistant bacteria due to misuse of antibiotics in medicine and livestock industry is causing serious problems for humans and livestock. Accordingly, strong regulations have been continuing from Jul. 1, 2011, in Korea, such as a total ban on addition of antibiotics in mixed feeds for animals. However, if antibiotics are not used, animals are directly exposed to diseases, and side effects may occur, such as a rapid increase in mortality rate or poor reproductive performance.

The below is the mechanism of action of antimicrobials, which are against viruses, bacteria, and the like, contained in a composition in which inorganic acids and organic acids which are components of ‘antibacterial and antiviral agent’ which are the background of the present invention, are mixed.

^{┌}Injection of composition ingredients in which organic and inorganic acids are mixed→Penetration into cell membranes of microorganisms and discharge of cell components→Destroy of microbial cells→Sterilization and antiviral action_{┘}

In particular, probiotics, which are microbial agents that have a beneficial effect on host animals by improving the bacterial colony of intestinal microorganisms, are used as one of the alternative antibiotics corresponding to antibacterial and antiviral agents. These probiotics are known to increase immunity to various diseases, reduce diarrhea, decrease serum cholesterol content, and enhance phagocytosis by white blood cells, and have advantages of inhibiting growths of other harmful microorganisms in the intestine and helping digestion and absorption of feeds.

In order to exert these effects inside the animal gastrointestinal tract, probiotics need to successfully settle in the intestinal tract; however, there is no clear evidence for this. Accordingly, there is a problem that probiotics must be continuously administered for the sustained beneficial effect. In addition, probiotics have a disadvantage that their efficacy is lower than that of conventional antibiotics, and the efficacy cannot be seen immediately.

In addition, plant extracts such as oriental herbal medicines have been effectively used in treatment of human infectious diseases as traditional Korean medicines with a long history. This plant extract has high advantages in that materials thereof have already been clinically secured and show an immediate effect. However, extracts from few plants such as saponins, herbs, and spice extracts are only partially used as alternatives to antibiotics, and systematic and scientific research for plant extracts are insufficient. In addition, storage problems have been reported due to characteristics of herbal medicines, and there is a disadvantage in that there is a palatability issue due to a bitter taste or an astringent taste.

In general, the antimicrobial spectrum of antimicrobial agents includes Gram-positive bacteria such as Bacillus cereus, Listeria monocytogenes, Mycobacterium spp., Staphylococcus aureus, Streptococcus spp., Pediococcus spp., and the like, Gram-negative bacteria such as E. Coli, Salmonella, Pseudomonas spp., Proteus spp., chicken necrotizing enteritis such as Cl. Perfringens and the like, viruses such as avian influenza (A.I.) virus, ND Virus, IB Virus (corona virus), pneumovirus, porcine PRRS Virus, Rota virus, porcine PED virus (corona virus), bovine viral diarrhea (BVD) virus, hepatitis B and hepatitis C virus, feline infectious peritonitis (corona virus), canine distemper virus, and the like.

However, even though herbal compositions may contribute to fermented products and antibacterial and immune enhancing compositions containing the fermented products that maximize an absorption rate and its efficacy in the body, effectively prevent diseases through a synergistic effect of various physiological actions, and have excellent antibacterial and immune enhancing effects while securing safety by using the fermentation technology utilizing active ingredients of the antimicrobial agent, research on those herbal compositions is still in the early stage and insufficient.

### PRIOR ART DOCUMENTS

### Patent Document

(Patent Document 0001) Korean Patent No. 10-1379404 (2014 Mar. 24)

### DETAILED DESCRIPTION OF THE INVENTION

### Technical Problems

First, “Impact” used as an antimicrobial agent of the present invention is defined. The Impact is a powerful antibacterial and antiviral alternative therapeutic agent formulated with a mixture of organic and inorganic acids and is said to be a new antibacterial and antiviral substitute for preventing and treating major diseases, such as viral, bacterial and fungal diseases, of livestock. The Impact is an absolutely necessary product for the production of eco-friendly livestock such as chicken and eggs and HACCP certified farms, which can be used for broiler chickens and finishing pigs that have diseases developed before market shipment in the case of no withdrawal period and laying hens during spawning.

The antimicrobial spectrum of the Impact of the antimicrobial agents mentioned above includes 1) Gram-positive bacteria such as Bacillus cereus, Listeria monocytogenes, Mycobacterium spp., Staphylococcus aureus, Streptococcus spp., Pediococcus spp., and the like, 2) Gram-negative bacteria such as E. Coli, Salmonella, Pseudomonas spp., Proteus spp., chicken necrotizing enteritis such as Cl. Perfringens and the like, 3) viruses including envelop viruses with lipophilic property such as avian influenza (A.I) virus, ND Virus, IB Virus (corona virus), pneumovirus, porcine PRRS Virus, Rota virus, porcine PED virus (corona virus), bovine viral diarrhea (BVD) virus, hepatitis B and hepatitis C virus, feline infectious peritonitis (corona virus), canine distemper virus, and the like.

An object of the present invention is to provide an antimicrobial agent composition, and a manufacturing method and a product thereof, which are formulated with organic acid with antimicrobial activity, carbonate compound, and chlorine based compound by utilizing natural extracts, thereby securing safety of fermentation technology and increasing its absorption rate and efficacy in the body to have an excellent antibacterial or antiviral immune enhancing effect through a synergistic effect of physiological action.

In addition, an object of a composition of the present invention is to achieve an antibacterial activity effect against major bacteria, which generally cause food poisoning, such as colon Bacillus and Salmonella, and Staphylococcus aureus, which causes skin suppuration, and to achieve an antiviral effect against envelop viruses such as herpesvirus, coronavirus, and influenza.

Furthermore, an object of an antimicrobial agent composition of the present invention is to manufacture a composite material such as natural extract and provide a antibacterial and antiviral substitute for preventing and treating major diseases, such as viral, bacterial and fungal diseases, of livestock and a product that is necessary for the production of eco-friendly livestock such as chicken and eggs and HACCP certified farms and that can be used for broiler chickens and finishing pigs that have diseases developed before market shipment in the case of no withdrawal period and laying hens during spawning.

### SUMMARY OF THE INVENTION

In the current situation where antibiotic resistance problems are caused by the misuse of antibiotics and the number of antibiotics that can treat diseases is decreasing, the present invention provides a composition using natural products and food additives having antibacterial and antiviral effects in order to improve public health.

In addition, the present invention is one of the methods for solving diseases such as SARS, MERS, and novel coronavirus which engulfs the whole world in fear.

An antimicrobial agent composition of the present invention is manufactured by selecting at least one or more of sodium chloride, calcium chloride, and potassium chloride, which are chlorides; selecting at least one or more of Pueraria lobata root extract, bellflower root extract, luffa extract, Portulaca extract, and citrus extract, which are plant extracts; selecting at least one or more of sodium carbonate, sodium bicarbonate, calcium carbonate, potassium carbonate, ammonium carbonate, and magnesium carbonate, which are carbonate compounds; selecting at least one or more of acetic acid and its related salts, lactic acid and its related salts, malic acid and its related salts, fumaric acid and its related salts, formic acid and its related salts, propionic acid and its related salts, succinic acid and its related salts, tartaric acid and its related salts, adipic acid and its related salts, pyruvic acid and its related salts, sodium hydrogen sulfate, and sulfonic acid and its related salts, which are organic acids; selecting a chloric acid based compound such as HClO, HClO₂, and HClO₃, which are inorganic acids; and mixing the selected ingredients with purified water.

The related salts mentioned above refer to sodium salts, potassium salts, calcium salts, and the like.

More specifically, an antimicrobial agent composition of the present invention, comprises an undiluted mixture solution formed by mixing: 1 to 10 weight % of a mixture comprising 50 to 60 weight % of purified water and chloride in which sodium chloride and calcium chloride which are at a 1:1 ratio, 50 weight %:50 weight %, are contained; 1 to 2 weight % of a mixture comprising Pueraria lobata root extract and bellflower root extract which are selected from among natural extracts and are at a 1:1 ratio, 50 weight %:50 weight %; 5 to 10 weight % of a mixture comprising carbonate based material in which sodium carbonate and sodium bicarbonate which are at a 1:1 ratio, 50 weight %:50 weight %, are contained; 10 to 15 weight % of a mixture comprising lactic acid and acetic acid among organic acids and their related salts, which are at a 1:1 ratio, 50 weight %:50 weight %; and 5 to 10 weight % of chloric acid based compound of inorganic acid.

In addition, 1 kg of the undiluted mixture solution may be diluted with 50 to 100 kg of purified water.

Furthermore, a method of manufacturing an antimicrobial agent of the present invention, comprises: a first step of selecting the antimicrobial agent composition of claim 1 or 2; a second step of converting the composition of the first step into a liquid product by applying heat to the composition at 50 to 60° C. and adding a solubilizer to completely dissolve the composition into liquefied composite material; a third step of forming the liquefied composite material into powder by heat-drying the liquefied composite material of the second step at 60° C.; and a fourth step of manufacturing a tablet product by shaping the heat-dried powder of the third step.

Moreover, a method of manufacturing an antimicrobial agent of the present invention, comprises: a first step of selecting the composition of claim 1 or 2; a second step of converting the composition of the first step into a liquid product by applying heat to the composition at 50 to 60° C. and adding a solubilizer to completely dissolve the composition into liquefied composite material; a third step of diluting 1 kg of the liquefied composite material of the second step with 50 to 100 kg of purified water.

In addition, the present invention provides a tablet product and a liquid product manufactured by the method of manufacturing an antimicrobial agent mentioned above.

### Technical Effects of the Invention

An antimicrobial agent composition, and its manufacturing method and product of the present invention are formulated with organic acid with antimicrobial activity, carbonate compound, and chlorine based compound by utilizing natural extracts, thereby having effects of securing safety of fermentation technology and increasing its absorption rate and efficacy in the body to have an excellent antibacterial or antiviral immune enhancing effect through a synergistic effect of physiological action.

In addition, a composition of the present invention has an antibacterial activity effect against major bacteria, which generally cause food poisoning, such as colon Bacillus and Salmonella, and Staphylococcus aureus, which causes skin suppuration, and an antiviral effect against envelop viruses such as herpesvirus, coronavirus, and influenza.

Furthermore, an antimicrobial agent composition and its manufacturing method and product of the present invention can be antibacterial and antiviral substitutes for preventing and treating major diseases of livestock, such as viral, bacterial and fungal diseases, can be products that are necessary for the production of eco-friendly livestock such as chicken and eggs and HACCP certified farms, and can be used for broiler chickens and finishing pigs that have diseases developed before market shipment in the case of no withdrawal period and laying hens during spawning.

### BRIEF DESCRIPTION OF THE DRAWINGS

FIG. 1 is a photograph of a result of a comparison test between the Impact (antimicrobial agent) of the present invention and ampicillin 100 (antibiotic) as a control material, against ATCC 6538 (Staphylococcus aureus) as shown in <Table 1>.

FIG. 2 is a photograph of a result of a comparison test between the Impact (antimicrobial agent) of the present invention and ampicillin 100 (antibiotic) as a control material, against ATCC 8739 (E. coli) as shown in <Table 1>.

FIG. 3 is a photograph of a result of a comparison test between the Impact (antimicrobial agent) of the present invention and ampicillin 100 (antibiotic) as a control material, against ATCC 14028 (Salmonella) as shown in <Table 1>.

FIGS. 4 and 5 are a test result report comparing the Impact (antimicrobial agent) of the present invention and the ampicillin 100 (antibiotic) as a control material.

### MODES FOR THE INVENTION

Hereinafter, in order to explain in detail to the extent that a person of ordinary skill in the art to which the present invention pertains can easily implement the technical idea of the present invention, specific embodiments of the present invention will be described as follows.

An antimicrobial agent composition of the present invention, comprises an undiluted mixture solution formed by mixing: 1 to 10 weight % of a mixture comprising 50 to 60 weight % of purified water and chloride in which sodium chloride and calcium chloride which are at a 1:1 ratio, 50 weight %:50 weight %, are contained; 1 to 2 weight % of a mixture comprising Pueraria lobata root extract and bellflower root extract which are selected from among natural extracts and are at a 1:1 ratio, 50 weight %:50 weight %; 5 to 10 weight % of a mixture comprising carbonate based material in which sodium carbonate and sodium bicarbonate which are at a 1:1 ratio, 50 weight %:50 weight %, are contained; 10 to 15 weight % of a mixture comprising lactic acid and acetic acid among organic acids and their related salts, which are at a 1:1 ratio, 50 weight %:50 weight %; and 5 to 10 weight % of chloric acid based compound of inorganic acid.

In addition, 1 kg of the undiluted mixture solution may be diluted with 50 to 100 kg of purified water.

Furthermore, a method of manufacturing an antimicrobial agent of the present invention, comprises: a first step of selecting the antimicrobial agent composition of claim 1 or 2; a second step of converting the composition of the first step into a liquid product by applying heat to the composition at 50 to 60° C. and adding a solubilizer to completely dissolve the composition into liquefied composite material; a third step of forming the liquefied composite material into powder by heat-drying the liquefied composite material of the second step at 60° C.; and a fourth step of manufacturing a tablet product by shaping the heat-dried powder of the third step.

Moreover, a method of manufacturing an antimicrobial agent of the present invention, comprises: a first step of selecting the composition of claim 1 or 2; a second step of converting the composition of the first step into a liquid product by applying heat to the composition at 50 to 60° C. and adding a solubilizer to completely dissolve the composition into liquefied composite material; a third step of diluting 1 kg of the liquefied composite material of the second step with 50 to 100 kg of purified water.

In addition, the present invention provides a tablet product and a liquid product manufactured by the method of manufacturing an antimicrobial agent mentioned above.

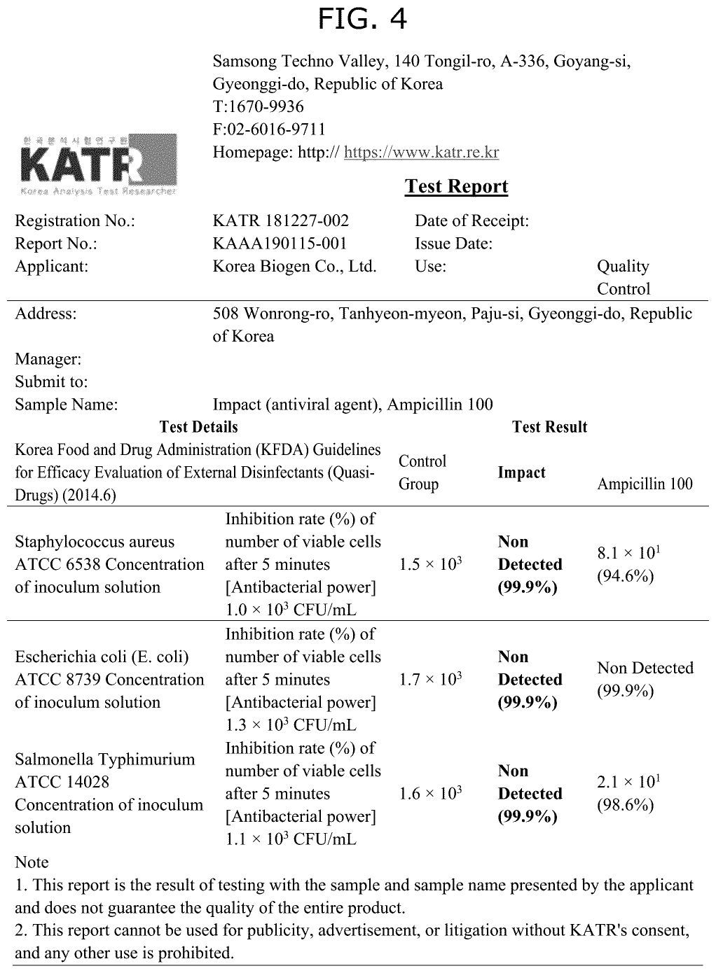

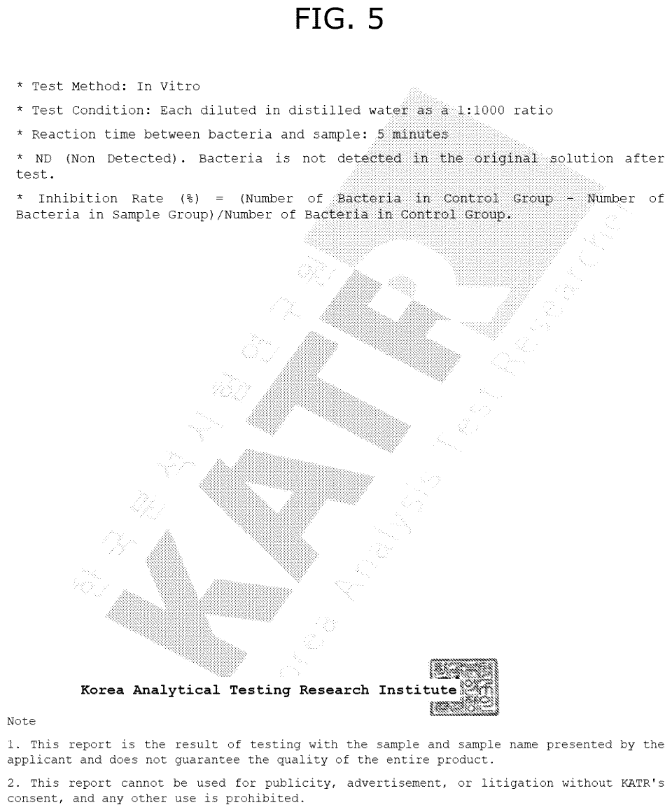

As shown in FIGS. 4 and 5, <Table 1> in the below is regarding a specific embodiment of the present invention and an official test report for the antimicrobial agent composition configured of an undiluted mixture solution formed by mixing: 1 to weight % of a mixture comprising 50 to 60 weight % of purified water and chloride in which sodium chloride and calcium chloride which are at a 1:1 ratio, 50 weight %:50 weight %, are contained; 1 to 2 weight % of a mixture comprising Pueraria lobata root extract and bellflower root extract which are selected from among natural extracts and are at a 1:1 ratio, 50 weight %:50 weight %; to 10 weight % of a mixture comprising carbonate based material in which sodium carbonate and sodium bicarbonate which are at a 1:1 ratio, 50 weight %:50 weight %, are contained; 10 to 15 weight % of a mixture comprising lactic acid and acetic acid among organic acids and their related salts, which are at a 1:1 ratio, weight %:50 weight %; and 5 to 10 weight % of chloric acid based compound of inorganic acid. The official test report shows the results of evaluating the Impact (antimicrobial agent) of the present invention through the antimicrobial agent composition against the three types of selected strains.

The <Table 1> below is based on the official test report of a test that was requested to and performed by the Korea Analytical Testing Research Institute as a test institution. The test was performed by an In vitro test method, and the <Table 1> shows test results of testing reaction states between the bacteria and the Impact sample for 5 minutes after diluting the Impact product to a prescribed concentration. In this case, an inhibition rate (%) of the test result is as follows: Inhibition Rate (%)=(Number of Bacteria in Control Group−Number of Bacteria in Sample Group)/Number of Bacteria in Control Group.

TABLE 1
Sample Name: Comparison Table of Impact (Antiviral
Agent) vs. Ampicillin 100 (Antibiotic)

| Test Details | | | | |
|---|---|---|---|---|
| Korea Food and Drug Administration | | | | |
| (KFDA) Guidelines for Efficacy | | Test Result | | |
| Evaluation of External Disinfectants | | Control | | Ampicillin |
| (Quasi-Drugs) (2014.6) | | Group | Impact | 100 |
| Staphylococcus | Inhibition rate | 1.5 × 10³ | Non | 8.1 × 10¹ |
| aureus | (%) of number | | Detected | (94.6%) |
| ATCC 6538 | of viable cells | | (99.9%) | |
| Concentration of | after 5 minutes | | | |
| inoculum | [Antibacterial | | | |
| solution | power] | | | |
| | 1.0 × 10³ | | | |
| | CFU/mL | | | |
| Escherichia coli | Inhibition rate | 1.7 × 10³ | Non | Non |
| (E. coli) | (%) of number | | Detected | Detected |
| ATCC 8739 | of viable cells | | (99.9%) | (99.9%) |
| Concentration of | after 5 minutes | | | |
| inoculum | [Antibacterial | | | |
| solution | power] | | | |
| | 1.3 × 10³ CFU/mL | | | |
| Salmonella | Inhibition rate | 1.6 × 10³ | Non | 2.1 × 10¹ |
| Typhimurium | (%) of number | | Detected | (98.6%) |
| ATCC 14028 | of viable cells after 5 minutes | | (99.9%) | |
| Concentration of | [Antibacterial | | | |
| inoculum | power] | | | |
| solution | 1.1 × 10³ CFU/mL | | | |

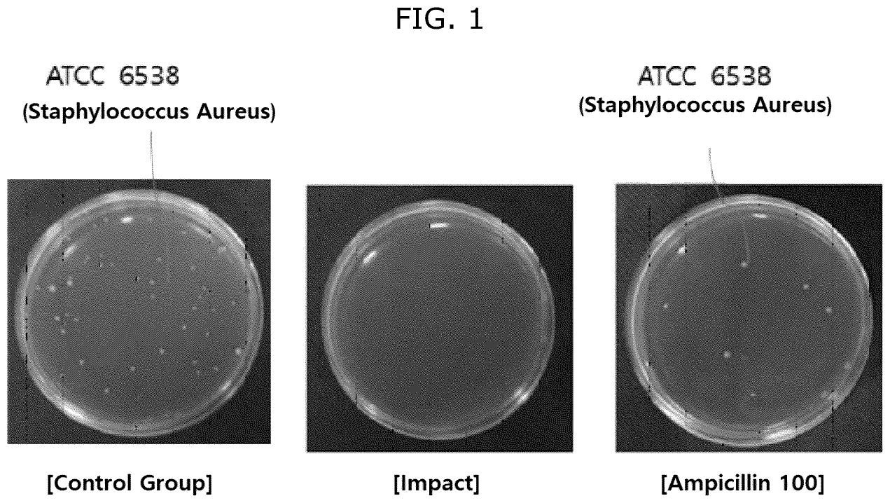

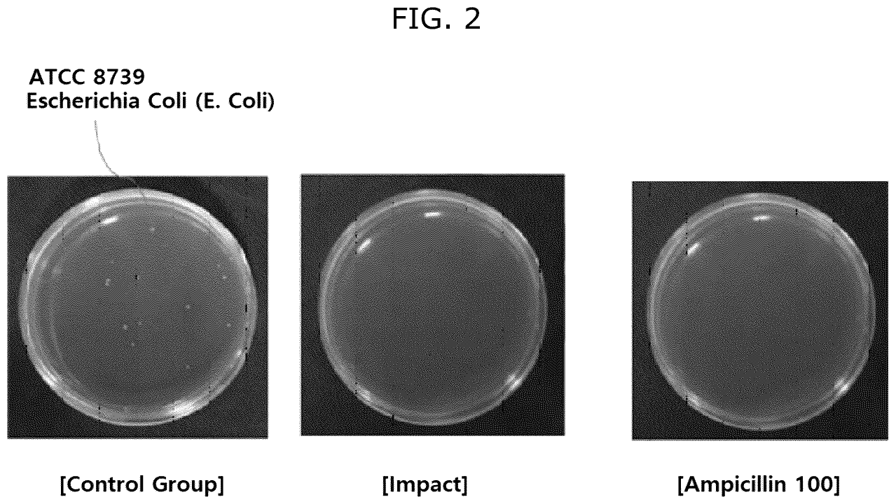

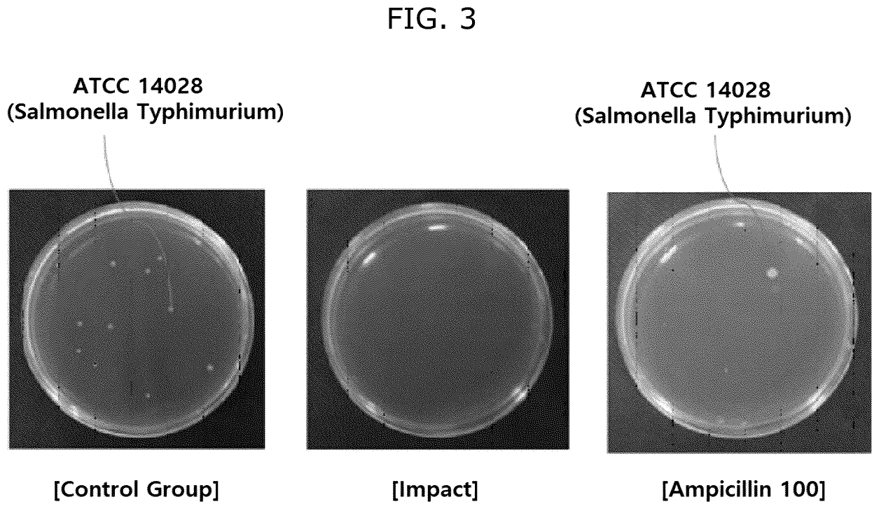

FIG. 1 is a photograph of a result of a comparison test between the Impact (antimicrobial agent) of the present invention and ampicillin 100 (antibiotic) as a control material, against ATCC 6538 (Staphylococcus aureus) as shown in <Table 1>. FIG. 2 is a photograph of a result of a comparison test between the Impact (antimicrobial agent) of the present invention and ampicillin 100 (antibiotic) as a control material, against ATCC 8739 (E. coli) as shown in <Table 1>. FIG. 3 is a photograph of a result of a comparison test between the Impact (antimicrobial agent) of the present invention and ampicillin 100 (antibiotic) as a control material, against ATCC 14028 (Salmonella) as shown in <Table 1>.

In the <Table 1>, 1) when a test item of Concentration of inoculum solution of Staphylococcus aureus ATCC 6538 was tested under a condition of an inhibition rate (%) of number of viable cells [Antibacterial power] 1.0×10³ CFU/mL after 5 minutes, the test result showed that 8.1×10¹ CFU/mL of bacteria (Inhibition Rate 94.6%) were detected in the ampicillin 100 group, but there was no bacteria detected in the Impact (antimicrobial agent) of the present invention (Inhibition Rate 99.9%). Accordingly, the test result shows an antibacterial effect of the present invention.

2) When a test item of Concentration of inoculum solution of Escherichia coli (E. coli) ATCC 8739 was tested under a condition of an inhibition rate (%) of number of viable cells [Antibacterial power] 1.3×10³ CFU/mL after 5 minutes, there were no bacteria detected both in the ampicillin 100 group (control material) and the Impact (antimicrobial agent) of the present invention.

3) When a test item of concentration of inoculum solution of Salmonella Typhimurium ATCC 14028 was tested under a condition of an inhibition rate (%) of number of viable cells [Antibacterial power] 1.1×10¹ CFU/mL after 5 minutes, the test result showed that 2.1×10¹ CFU/mL of bacteria (Inhibition Rate 98.6%) were detected in the ampicillin 100 group, but there were no bacteria detected in the Impact (antimicrobial agent) of the present invention (Inhibition Rate 99.9%). Accordingly, the test result shows an antibacterial effect of the present invention.

The present invention was clinically tested to each of groups divided into broiler chicken, broiler chicken breeding, laying hen, pigs, dog, cat, and human (simple clinical test) as follows.

First, the below are an administration test method and a result for broiler chicken.

In the first test, referring to <Table 2> in the below, Escherichia coli was diagnosed among 30,000 of Ross breeds at the age of 25 days at a broiler chicken consignment farm in Miyang-myeon, Anseong-si, Gyeonggi-do, Korea, and sepsis and mortality occurred. For the test, the product of the present invention was diluted in drinking water at an appropriate ratio and fed for 3 days. As a result of the test, the mortality disappeared after the administration, the symptoms improved, and feed intake was restored to normal.

TABLE 2
Administration Test to Broiler Chicken - 1

| Test | |
|---|---|
| Information | Test Result |
| Farm Name | Broiler chicken consignment farm |
| | [Breed: Ross, Age: 25 days, Population: 30,000] |
| Used Product | Impact |
| Administration | Escherichia coli was diagnosed at the age of 25 |
| Method | days, and sepsis and mortality occurred. The |
| | product of the present invention was diluted as |
| | a 1:1000 ratio in drinking water and fed for 3 |
| | days |
| Test Result | After the administration, the mortality |
| | disappeared, the symptoms improved, and feed |
| | intake was restored to normal. |

In the second test, referring to <Table 3> in the below, infectious bronchitis (corona virus) occurred among 24,000 of Ross breeds at the age of 20 days at the broiler chicken consignment farm mentioned above. The Ross breeds showed initial respiratory symptoms, and feed intake was reduced. Antibiotic (Ampicillin) was administered, but respiratory symptoms persisted. For the test, the product of the present invention was fed at the recommended dosage and was administered in the same way for 3 days. As a result of the test, symptoms improved the next day after the first administration, respiratory symptoms disappeared after administration for 3 days, and feed intake was normalized.

TABLE 3
Administration Test to Broiler Chicken - 2

| Test | |
|---|---|
| Information | Test Result |
| Farm Name | Broiler chicken consignment farm |
| | [Breed: Ross, Age: 20 days, Population: 24,000] |
| Location | Miyang-myeon, Anseong-si, Gyeonggi-do, Korea |
| Situation | Infectious bronchitis (corona virus) occurred |
| Related to | among 24,000 of Ross breeds at the age of 20 days. |
| Disease | The Ross breeds showed initial respiratory |
| | symptoms, and feed intake was reduced. Antibiotic |
| | (Ampicillin) s administered, but respiratory |
| | symptoms persisted. |
| Administration | The 1 L of “Impact” of the present invention was |
| Method | diluted in 1 ton of drinking water for 10,000 to |
| | 15,000 and fed for a day and was administered in |
| | the same way for 3 days. |
| Test Result | After the first administration, symptoms improved |
| | the next day; respiratory symptoms disappeared |
| | after administration for 3 days; and feed intake |
| | was normalized. |

In the third test, referring to <Table 4> in the below, Salmonella occurred among 55,000 of Ross breeds at the Seonghwa foods broiler consignment farm in Susin-myeon, Dongnam-gu, Cheonan-si, Korea, and an average number of mortalities per day was approximately 240. In addition, at the age of 10 days, a total of 2,600 have been killed. For the test, at 12 days of age, the product of the present invention was fed at the recommended dosage for 3 days. As a result of the test, mortality decreased sharply to 50 per day, and drinking water consumption increased. In addition, at the time of shipment, a growth rate was 95.5%, and a weight was 1.6 kg, which was good.

TABLE 4
Breeding Test to Broiler Chicken - 3

| Test | |
|---|---|
| Information | Test Result |
| Farm Name | Seonghwa Foods Broiler Consignment Farm |
| | [Breed: Ross, Population: 55,000] |
| Location | Susin-myeon, Dongnam-gu, Cheonan-si, Korea |
| Used Product | Impact |
| Disease/ | Salmonella occurred; an average number of |
| Administration | mortalities per day was approximately 240; and |
| Method | at the age of 10 days, a total of 2,600 have |
| | been killed. |
| | At 12 days of age, the product of the present |
| | At 12 days of age, the product of the |
| | invention was diluted at a ratio of 1 L of the |
| | product to 1 ton of drinking water and fed for |
| | 3 days. |
| Test Result | After the administration of the “Impact,” |
| | mortality decreased sharply to 50 per day; and |
| | drinking water consumption increased. At the |
| | time of shipment, a growth rate was 95.5%, and |
| | a weight was 1.6 Kg, which was good. |

In the fourth test, referring to <Table 5> in the below, a laying hen test 1 was conducted. At the TY farm located in Yeoncheon-gun, Gyeonggi-do, Korea, infectious bronchitis (corona virus) occurred among 2,500 of High-line breeds. A laying rate decreased along with a mortality increase. For the test, the product of the present invention was continuously fed for 5 days at the recommended dosage. The mortality stopped 3 days after administration, an occurrence of abnormal eggs decreased, and the egg laying rate gradually recovered.

TABLE 5
Test to Laying Hen - 1

| Test | |
|---|---|
| Information | Test Result |
| Farm Name | TY farm |
| | [Breed: High Line, Population: 25,000] |
| Location | Yeoncheon-gun, Gyeonggi-do, Korea |
| Used Product | Impact (Antibacterial and antiviral |
| | substitute) |
| Disease/ | Infectious bronchitis (corona virus) occurred, |
| Administration | and a laying rate decreased along with a |
| Method | mortality increase. |
| | The product of the present invention was diluted |
| | at a ratio of 1 L of the product to 1 ton of |
| | drinking water and fed for 5 days. |
| Test Result | After 3 days after the administration, the |
| | mortality stopped; an occurrence of abnormal |
| | eggs decreased; and the egg laying rate |
| | gradually recovered. |

In the fifth test, referring to <Table 6> in the below, a laying hen test 2 was conducted. At the TY farm located in Ipjyang-myeon, Cheonan-si, Korea, inclusion body hepatitis (adeno virus) occurred among 70,000 of High Line breeds 7, and an average number of mortalities per day was approximately 500 from the age of 12 weeks. For the test, from the second day after the occurrence, the product of the present invention was fed at the recommended dose for 5 days. After administration of this product, mortality rapidly decreased to 50 per day, and from the third day after administration, mortality completely disappeared and returned to normal.

TABLE 6
Test to Laying Hen - 2

| Test | |
|---|---|
| Information | Test Result |
| Farm Name | K farm |
| | [Breed: High Line, Population: 70,000] |
| Location | Ipjyang-myeon, Cheonan-si, Korea |
| Used Product | Impact |
| Disease/ | Inclusion body hepatitis (adeno virus) occurred, |
| Administration | and an average number of mortalities per day was |
| Method | approximately 500 from the age of 12 weeks. |
| | Since the second day after the occurrence, |
| | Newcosin was diluted at a ratio of 1 L of the |
| | product to 1 ton of drinking water and fed for |
| | 5 days. |
| Test Result | After the administration of the present product, |
| | mortality rapidly decreased to 50 per day; and |
| | from the third day after administration, |
| | mortality completely disappeared and returned to |
| | normal. |

In the sixth test, referring to <Table 7> in the below, a pig test 1 was conducted. At a finisher pig farm located in Icheon-si, Gyeonggi-do, Korea, respiratory and reproductive syndrome (PRRS virus) occurred among 1000 breeding pigs of a LDY hybrid breed, which were at the age of 2 to 3 months and weighted 30-40Kg. For the test, the product of the present invention was fed at the recommended dose and fed for 5 days in the same way. As a result of the test, the symptoms improved after 5 days of administration, the respiratory symptoms disappeared from the third day of the administration, and feed intake was normalized.

TABLE 7
Administration Test to Pig - 1

| Test | |
|---|---|
| Information | Test Result |
| Farm Name | Finisher pig farm |
| | [Breed: LDY hybrid, Age: 2 to 3 months, |
| | Population: 1,000] |
| Location | Icheon-si, Gyeonggi-do, Korea |
| Situation | Respiratory and reproductive syndrome (PRRS |
| Related to | virus) occurred among breeding pigs with 30- |
| Disease | 40 Kg of weight. |
| Administration | The 1,000 mL of the “Impact” of the present |
| Method | invention was diluted in 1 ton of drinking water |
| | and fed daily for 5 days. |
| Test Result | After 5 days of the administration, the symptoms |
| | were improved; the respiratory symptoms |
| | disappeared from the third day of the |
| | administration; and feed intake was normalized. |

In the seventh test, referring to <Table 8> in the below, the pig test 2 was conducted. At a finisher pig farm located in Nonsan-si, Chungcheongnam-do, Korea, E. coli diarrhea occurred among 1,000 of weaned pigs of a LDY hybrid, which were weighted 10 to 20 kg. The product of the present invention was fed at the recommended dosage and was fed for 5 days in the same way. After 3 days of the administration, as a result of the test, diarrhea stopped, and symptoms gradually improved.

TABLE 8
Administration Test to Pig - 2

| Test | |
|---|---|
| Information | Test Result |
| Farm Name | Finisher pig farm |
| | [Breed: LDY hybrid, Population: 5,000] |
| Location | Nonsan-si, Chungcheongnam-do, Korea |
| Situation | E. coli diarrhea occurred among 1,000 of weaned |
| Related to | pigs, which were weighted 10 to 20 kg. |
| Disease | |
| Administration | The 1 L of the “Impact” of the present invention |
| Method | was diluted in 1 ton of drinking water and fed |
| | daily for 5 days. |
| Test Result | After 3 days of the administration, diarrhea |
| | stopped; and symptoms gradually improved. |

In the eighth test, referring to <Table 9> in the below, the pig test 3 was conducted. At a batch breeding farm located in in Hwaseong-si, Gyeonggi-do, Korea, white diarrhea (E. coli and rotavirus) occurred among weaned pigs from 200 of brood sows of a LDY hybrid. For each weaned pig, 2 mL of the product of the present invention was diluted in 100 mL of drinking water, orally administrated, and fed for 5 days in the same way. As a result of the test, diarrhea stopped from the next day after the first administration.

TABLE 9
Administration Test to Pig - 3

| Test | |
|---|---|
| Information | Test Result |
| Farm Name | Batch breeding farm |
| | [Breed: LDY hybrid, Population: 200] |
| Location | Hwaseong-si, Gyeonggi-do, Korea |
| Situation | White diarrhea (E. coli and rotavirus) |
| Related to | occurred. |
| Disease | |
| Administration | For each weaned pig, 2 mL of the product of the |
| Method | present invention was diluted in 100 mL of |
| | drinking water, orally administrated, and fed for |
| | 5 days in the same way. |
| Test Result | From the next day after the first administration, |
| | diarrhea stopped. |

In the ninth test, referring to <Table 10> in the below, the pig test 4 was conducted. At a batch breeding farm located in Haman-gun, Gyeongnam-do, Korea, porcine epidemic diarrhea virus PED (corona virus) occurred among both suckling pigs and 200 of brood sows of a LDY hybrid. For each suckling pig, 2 mL of the product of the present invention was diluted in 100 mL of drinking water and orally administered, and for brood sows and growing pigs were fed at an appropriate dose for 5 days. As a result of the test, diarrhea started to stop on the third day after the administration, and the pigs were recovered after 5 days.

TABLE 10
Administration Test to Pig - 4

| Test | |
|---|---|
| Information | Test Result |
| Farm Name | Batch breeding farm |
| | [Breed: LDY hybrid, Population: 200] |
| Location | Haman-gun, Gyeongnam-do, Korea |
| Situation | Porcine epidemic diarrhea virus PED (corona |
| Related to | virus) occurred among both suckling pigs and |
| Disease | brood sows. |
| Administration | For each weaned pig, 2 mL of the “Impact” was |
| Method | diluted in 100 mL of drinking water, and orally |
| | administrated for 5 days. For each brood sow and |
| | growing pig, the “Impact” was diluted at a ratio |
| | of 1 L of the product to 1 ton of drinking water, |
| | and consistently fed for 5 days. |
| Test Result | From the third day after the administration, |
| | diarrhea started to stop, and the pigs were |
| | recovered after 5 days. |

In the tenth test, referring to <Table 11> in the below, the cat test 1 was conducted. Koshort breeds, which were at the age of 7 months and weighted 2.7 kg, from Bubal-eup, Icheon-si, Gyeonggi-do, Korea, visited the hospital due to bloody diarrhea, and a positive result for feline leukopenia (FP virus) was obtained in the kit test. The blood test showed a 0 count of lymphocyte, a 1.3×10³/ul count of white blood cell, and a 41×10³/ul count of platelets, which were significant low levels. Therefore, for the test, 1 mL of the product of the present invention was orally administrated daily for each cat, and after a day from the oral administration, 0.3 mL of the product was administrated every day. As a result of the test, bloody stools stopped 3 days after the administration, but appetite was still poor. The blood test after getting an IV (intravenous injection) showed that a count of lymphocyte was recovered to 2.7, which was normal; a count of white blood cell slightly increased to 38, and a count of platelet was recovered to 333, which was normal.

TABLE 11
Administration Test to Cat - 1

| | | |
|---|---|---|
| Species: Cat | Breed: Koshort | Age: 7 months |
| | Sex: Female (neutered) | |

| | |
|---|---|
| Weight: 2.7 kg | Address of Owner: Bubal-eup, Icheon-si, |
| | Gyeonggi-do, Korea |

Disease
Feb. 6, 2020:
Visited with bloody diarrhea
A positive result for feline leukopenia (FP virus) was
obtained in the kit test.
The blood test showed a 0 count of lymphocyte, a 1.3 × 10³/
ul count of white blood cell, and a 41 × 10³/ul count of
platelets, which were significant low levels.
Administration
Feb. 6, 2020: Administration of fluids at the same time as
hospitalization. Injection of antiserum injection.
Feb. 7, 2020: Oral administration of 1 mL of an antiviral agent
with no sweeting agents for each cat. After the administration,
cat showed vomiting symptoms.
Feb. 8, 2020 to Feb. 13, 2020: Oral administration of 0.3 mL of
Impact containing a sweeting agent every day.
Test Result
After 3 days of the administration, bloody stools stopped, but
appetite was still poor. Administration of an IV (intravenous
injection).
Feb. 13, 2020: The blood test showed that a count of lymphocyte
was recovered to 2.7, which was normal; a count of white blood
cell slightly increased to 38, and a count of platelet was
recovered to 333, which was normal.

In the eleventh test, referring to <Table 12> in the below, the cat test 2 was conducted. A Koshort breed, which was at the age of 6 months and weighted 2.3 kg, from Baeksa-myeon, Icheon-si, Gyeonggi-do, Korea, visited the hospital with a respiratory problem and tentatively determined as feline respiratory disease complex (FRDC) based on sleep discharge and the respiratory problem.

On Feb. 6, 2020, antibiotics and anti-inflammatory drugs were prescribed, but 6 out of 10 died within 3 days. For the test, 0.2 mL of the “Impact”, which was mixed with sugar, of the present invention was orally administrated for each cat from Feb. 10, 2020 to Feb. 14, 2020. As a result of the test, the remaining 4 of cats all become healthy without death and were active normally.

TABLE 12
Administration Test to Cat - 2

| | | |
|---|---|---|
| Species: Cat | Breed: Koshort | Age: 6 months |
| Count: 10 stray cats | Address of Owner: Baeksa-myeon, | |
| Weight: 2.3 kg on | Icheon-si, Gyeonggi-do, Korea | |
| average | | |

Disease:
Feb. 6, 2020:
Visited with a respiratory problem.
Tentatively determined as feline respiratory disease
complex (FRDC) based on sleep discharge and the respiratory
problem.
Administration
Feb. 6, 2020: Antibiotics and anti-inflammatory drugs were
prescribed, but 6 out of 10 died within 3 days.
Feb. 10, 2020 to Feb. 14, 2020: Oral administration of 0.2 mL of
Impact containing sugar for each cat.
Test Result
The remaining 4 of cats all become healthy without death and
were active normally.

In the twelfth test, referring to <Table 13> in the below, the dog test was conducted. A mixed breed, which was male at the age of 1 year and 1 month and weighted 6 kg, from Aeryeonjeong-ro, Icheon-si, Gyeonggi-do, Korea, visited to the hospital with continuous cough, high fever of 40° C., vomiting, and diarrhea and was hospitalized with interstitial pneumonia based on X-ray and examination.

On the first day, antipyretic, anti-serum, antibiotic, and fluid were administered, and for 3 days thereafter, 0.2 mL of antiviral agent mixed with sugar water was orally administered for each cat. As a result of the test, symptoms improved, and he was discharged from the hospital.

TABLE 13
Administration Test to Dog

| | | |
|---|---|---|
| Species: Dog | Breed: | Age: 1 year and |
| | Mixed | 1 month |
| | Sex: Male | |

| | |
|---|---|
| Weight: 6 kg | Address of Owner: |
| | Aeryeonjeong-ro, Icheon-si, |
| | Gyeonggi-do, Korea, |

Disease:
Jan. 24, 2020: Visited to the hospital with continuous cough, high
fever of 40° C., vomiting, and diarrhea and was hospitalized with
interstitial pneumonia based on X-ray and examination.
Administration
First day: Antipyretic, anti-serum, antibiotic, and fluid were
administered.
3 Days after the first day: 0.2 mL of antiviral agent mixed
with sugar water was orally administered.
Test Result
The symptoms improved, and he was discharged from the hospital.

In the thirteenth test, efficacy was tested on humans as a simple clinical trial, and the following results were obtained.

[Case 1]

Name: Jo, xxx (male, 54 years old), living in Jeungpo-dong, Icheon-si, Korea infection

Symptoms: sore throat, high fever, and cold virus infection

Treatment: After administration of 1 mL of the antibacterial antiviral agent per day for 3 days, the symptoms of cold completely disappeared.

[Case 2]

Name: Kim, xxx (female, 63 years old), living in Taepyeong-dong, Seongnam-si, Korea

Symptoms: Cold symptoms. Even though a cold medicine was administrated for 7 days, the symptoms did not get better.

Treatment: After administration of 1 mL of the antibacterial and antiviral agent per day for 2 days, the cold was disappeared.

[Case 3]

Name: Park, xxx (male, 49 years old), living in Changjeon-dong, Icheon-si, Korea

Symptoms: sore throat, fever, and cold symptoms.

Treatment: After administration of 1 ml of antibacterial antiviral agent per day for 3 days, the cold was disappeared.

[Case 4]

Name: Kim, xxx (male, 27 years old), living in Bundang-gu, Seongnam-si, Korea

Symptoms: Sore throat and cold symptoms

Treatment: 1 mL of the antibacterial and antiviral agent was administered for 2 days, and then the cold was disappeared.

As described above, although the present invention has been shown and described in relation to the specific embodiments described above, various modifications and changes are possible without departing from the spirit and scope of the invention as indicated by the following claims. Anyone with ordinary knowledge in the relevant technical field will be able to easily understand.

1. An antimicrobial agent composition comprising an undiluted mixture solution formed by mixing:
1 to 10 weight % of a mixture comprising 50 to 60 weight % of purified water and chloride in which sodium chloride and calcium chloride which are at a 1:1 ratio, 50 weight %:50 weight %, are contained;
1 to 2 weight % of a mixture comprising Pueraria lobata root extract and bellflower root extract which are selected from among natural extracts and are at a 1:1 ratio, 50 weight %:50 weight %;
5 to 10 weight % of a mixture comprising carbonate based material in which sodium carbonate and sodium bicarbonate which are at a 1:1 ratio, 50 weight %:50 weight %, are contained;
10 to 15 weight % of a mixture comprising lactic acid and acetic acid among organic acids and their related salts, which are at a 1:1 ratio, 50 weight %:50 weight %; and
5 to 10 weight % of chloric acid based compound of inorganic acid.

2. The antimicrobial agent composition of claim 1, wherein 1 kg of the undiluted mixture solution is diluted with 50 to 100 kg of purified water.

3. A method of manufacturing an antimicrobial agent, the method comprising:
a first step of selecting the antimicrobial agent composition of claim 1;
a second step of converting the composition of the first step into a liquid product by applying heat to the composition at 50 to 60° C. and adding a solubilizer to completely dissolve the composition into liquefied composite material;
a third step of forming the liquefied composite material into powder by heat-drying the liquefied composite material of the second step at 60° C.; and
a fourth step of manufacturing a tablet product by shaping the heat-dried powder of the third step.

4. A method of manufacturing an antimicrobial agent, the method comprising:
a first step of selecting the composition of claim 1;
a second step of converting the composition of the first step into a liquid product by applying heat to the composition at 50 to 60° C. and adding a solubilizer to completely dissolve the composition into liquefied composite material;
a third step of diluting 1 kg of the liquefied composite material of the second step with 50 to 100 kg of purified water.

5. A tablet product manufactured by the method of manufacturing an antimicrobial agent according to claim 3.

6. A tablet product manufactured by the method of manufacturing an antimicrobial agent according to claim 4.

7. A method of manufacturing an antimicrobial agent, the method comprising:
a first step of selecting the antimicrobial agent composition of claim 2;
a second step of converting the composition of the first step into a liquid product by applying heat to the composition at 50 to 60° C. and adding a solubilizer to completely dissolve the composition into liquefied composite material;
a third step of forming the liquefied composite material into powder by heat-drying the liquefied composite material of the second step at 60° C.; and
a fourth step of manufacturing a tablet product by shaping the heat-dried powder of the third step.

8. A method of manufacturing an antimicrobial agent, the method comprising:
a first step of selecting the composition of claim 2;
a second step of converting the composition of the first step into a liquid product by applying heat to the composition at 50 to 60° C. and adding a solubilizer to completely dissolve the composition into liquefied composite material;
a third step of diluting 1 kg of the liquefied composite material of the second step with 50 to 100 kg of purified water.

9. A tablet product manufactured by the method of manufacturing an antimicrobial agent according to claim 7.

10. A tablet product manufactured by the method of manufacturing an antimicrobial agent according to claim 8.
